(19) United States
(12) Patent Application Publication
(10) Pub. No.: US 2026/0085089 A1
(43) Pub. Date: Mar. 26, 2026

(54) CONTINUOUS GRADIENT ELUTION CHROMATOGRAPHIC FRACTIONATION

(71) Applicant: SANOFI AVENTIS DEUTSCHLAND GMBH, Frankfurt am Main (DE)

(72) Inventors: Oliver BOSCHEINEN, Frankfurt am Main (DE); Matthias GUNNE, Frankfurt am Main (DE); Daniel SCHEPS, Frankfurt am Main (DE); Jochen STRUBE, Clausthal (DE); Steffen ZOBEL-ROOS, Clausthal (DE)

(21) Appl. No.: 19/111,122

(22) PCT Filed: Sep. 12, 2023
(86) PCT No.: PCT/EP2023/074950

§ 371 Date: Mar. 12, 2025

(30) Foreign Application Priority Data
Sep. 13, 2022 (EP) 22195403.5

(51) Int. Cl. C07K 1/22; B01D 15/16; B01D 15/18; B01D 15/24; B01D 15/32; B01D 15/42

(52) U.S. Cl. CPC C07K 1/22; B01D 15/166; B01D 15/1864; B01D 15/247; B01D 15/325; B01D 15/424

(57) ABSTRACT

The present invention relates to a method for separating a product of interest from impurities and an apparatus for performing the method. The method comprises the steps in the indicated order: (1) loading on a chromatography matrix a first volume of a feed solution comprising the product of interest and impurities; (2) contacting the chromatography matrix with an elution solution; (3) collecting a) optionally an elution fraction 1 (EF1) in a side fraction container (SFC), b) an elution fraction 2 (EF2) in a product container, and c) optionally an elution fraction 3 (EF3) in a SFC, wherein at least one of EF1 and EF3 is collected; (4) loading on a chromatography matrix EF1 and/or EF3 and a second volume of the feed solution simultaneously or subsequently. Steps (2) to (4) are repeated at least once, and the chromatography matrices of step (1) and (4) are the same or different.

### TECHNICAL FIELD OF THE INVENTION

The present invention relates to the field of chromatography. More specifically, the present invention relates to a continuous gradient elution chromatographic fractionation method, allowing highly effective separation and purification of products in solution from impurities.

### BACKGROUND OF THE INVENTION

Chromatography is a widely used unit operation for analytical as well as preparative separations, especially to produce high purity products like pharmaceuticals. In these cases, the desired target component is often eluting closely with impurities, as depicted in FIG. 1. To achieve high purity, narrow product fractionation is one of the main goals. A narrow product fractionation, however, leads to product loss since the target product often elutes with impurities in a bell-shaped manner. It is thus one aim of the present invention to reduce this loss.

Several chromatography methods are known in the art. Specifically continuous chromatography is described in Subramanian (Continuous biomanufacturing. Innovative Technologies and Methods. 2018, Weinheim: WILEY-VCH), Zobel-Roos (Entwicklung, Modellierung und Validierung von integrierten kontinuierlichen Gegenstrom-Chromatographie-Prozessen. 1. Auflage, 2018. Herzogenrath: Shaker (Thermische Verfahrens-und Prozesstechnik), Schmidt-Traub et al. (Preparative chromatography. 2., completely revised and updated ed. 2012, Weinheim, Germany: WILEY-VCH). Continuous chromatography includes several sub-categories such as (i) simulated moving bed chromatography (SMB) described in Imamoglu (Simulated moving bed chromatography (SMB) for application in bioseparation. Advances in biochemical engineering/biotechnology 2105, Vol. 76, p. 211-231), Rodrigues (Simulated Moving Bed Technology. Principles, Design and Process Applications. 2015, Burlington: Elsevier Science), (ii) sequential chromatography described in Holzer (Sequential Multi-Column Chromatography. BPI. Düsseldorf, April 2013), Bisschops (BioSMB™ Technology: Continuous Countercurrent Chromatography Enabling a Fully Disposable Process. Ganapathy Subramanian (Hg.): Biopharmaceutical Production Technology. 1^{st} ed. 2012, Weinheim: WILEY-VCH, p. 769-791), Whitford (Single-Use Systems As Principal Components in Bioproduction. BioProcess International 2010, Vol. 8 (11), p. 34-44), Angarita et al. (Twin-column CaptureSMB: a novel cyclic process for protein A affinity chromatography. Journal of chromatography, Vol. 1389, 2015, p. 85-95), and in Godawat et al. (Periodic counter-current chromatography-design and operational considerations for integrated and continuous purification of proteins. Biotechnology Journal 2012, Vol. 7 (12), p. 1496-1508), and (iii) multicolumn countercurrent solvent gradient purification (MCSGP) described in Aumann und Morbidelli (A continuous multicolumn countercurrent solvent gradient purification (MCSGP) process. Biotechnology and Bioengineering 2007, Vol. 98 (5), p. 1043-1055); Müller-Späth und Morbidelli (Continuous Chromatography for the Purification of Monoclonal Antibodies. Uwe Gottschalk (Hg.): Process scale purification of antibodies. Hoboken, N.J.: John Wiley & Sons, 2009, p. 223-238), and in Steinebach et al. (Continuous counter-current chromatography for capture and polishing steps in biopharmaceutical production. Biotechnology Journal 2016, Vol. 11 (9), p. 1126-1141).

Specifically, multicolumn countercurrent solvent gradient purification (MCSGP) is a process that was initially conceived for continuous centre-cut operations in gradient chromatography (Aumann & Morbidelli 2007, Aumann et al. (Parametric study of a 6-column countercurrent solvent gradient purification (MCSGP) unit. Biotechnology and Bioengineering, 2007, Vol. 98 (5), p. 1029-1042), Ströhlein et al. (A continuous, counter-current multi-column chromatographic process incorporating modifier gradients for ternary separations. Journal of chromatography, 2006, Vol. 1126(1-2), p. 338-346); Müller-Späth (Purification of monoclonal antibodies by continuous chromatography. Zugl.: Zürich, Diss., Eidgenössische Technische Hochschule ETH Zürich, Nr. 18066, 2009). As shown in FIG. 2, three columns are operated separately, and three columns are interconnected. The solo columns are eluting pure product (red) and the strong (green) and weak (blue) binding side components. The interconnected columns are separating the overlapping regions of product and impurities. After a given time, each column is switched one position to the left. In the connected line, the column to the left is eluted with the highest elution strength, the column in the middle with a lower elution strength and the column on the right is in equilibration state. The components are flowing from one column to the other where they are meant to bind again. To do so, the elution strength of the liquid phase needs to be lower between the columns. Thus, the streams are diluted.

In an improved variant of this method, the 6-column setup as shown in FIG. 2 was revised and adapted to a twin-column setup (Aumann und Morbidelli 2008; Krattli et al. (Online control of the twin-column countercurrent solvent gradient process for biochromatography. Journal of chromatography. Vol. 1293, 2013, p. 51-59); Krättli et al. (Closed loop control of the multi-column solvent gradient purification process. Journal of chromatography, 2011, Vol. 1218 (50), p. 9028-9036); Müller-Späth et al. 2013; Steinebach et al. 2016). This twin-column setup requires eight steps, which are shown in FIG. 3. In the first step, the cycle starts with loading the overlap of weak binding component (W) and product (P) from column 1 to column 2. To account for the elution strength of the fraction (e.g. the amount of salt or modifier such as a polar solvent), the feed is diluted in-line with pure eluent (E). In the second step, when purity criteria are met, product (P) is gained from column 1, and column 2 is loaded with feed. In the third step, when the overlap of product and strong binding impurity(S) is about to elute, column 1 is again connected to column 2 and the fraction is diluted in-line with eluent (E). In the fourth step, the gradient for column 2 is started, and weak binding components (W) are eluted and transferred to waste. The gradient for column 1 comes to the end, and strong binding impurities(S) are discharged. This process is repeated once but with switched roles for columns 1 and 2 (steps 5 to 8). The choice of cut points and the in-line dilution are critical for this process. In addition, the columns must be synchronized. Feed loading and product elution (see step 2 or 6) must occur at the same time and should take the same amount of time. Otherwise, the impurities would be shifted. It is intended that the weak binding impurity from column 1 is loaded to column 2 before feed loading and the strong binding component after feed loading.

The methods of the prior art and specifically the two-column MCSGP process have clear disadvantages. First, there is no continuous feed loading. A continuous purification process is usually defined by at least one continuous stream. In the twin-column MCSGP, for example, both streams are discontinuous and feed loading and product elution (red arrows in FIG. 4) take place in steps 2 and 6 only. Note that the eluted product is not loaded to the other column. Second, there is a lack of synchronization between the individual steps as depicted in FIG. 4. For an ideal twin-column MCSGP process, feed loading on one column and the gradient on the other column should be synchronized and both steps should take the same amount of time. In many cases, however, one step takes by far longer than the other. Especially the step of feed loading usually takes much longer than the elution step. Methods therefore often show a loading step requiring 5 to 10 times as long as the elution step. Therefore, steps 2 and 6 will take as long as it needs to load a column. In the meantime, the other column can only perform product elution with very low flow rates, or it can perform product elution with the ideal speed but then has to be stopped. Both is disadvantageous due to e.g. diffusional processes. A similar problem occurs after steps 4 and 8. After the gradient separation, each column is going through a high elution regeneration followed by a re-equilibration (compare blue and yellow lines in FIG. 4). This re-equilibration must be completed before loading the weak binding overlap (step 1 and 5). Thus, the other column has to wait again. Due to very long column loading compared to the time needed to elute the product, classical MCSGP process show relatively low productivity. In summary, one column has to wait with its gradient until the other column is loaded completely, shortly after the same column forces the other to wait until it completes the post-elution steps. This procedure is highly ineffective and time consuming.

Thus, there is a need for novel chromatography methods which overcome the disadvantages of the prior art, such as slow and ineffective methods and unwanted diffusion processes in the columns that are not fed and not eluted (i.e. put on hold). There is further a need for novel chromatography methods which reduce the loss of the product.

### SUMMARY OF THE INVENTION

The invention provides in a first aspect, the present invention provides a method of separating a product of interest from impurities. The method comprises the following steps in the indicated order:
(1) loading on a chromatography matrix a first volume of a feed solution comprising the product of interest and impurities;
(2) contacting the chromatography matrix with an elution solution;
(3) collecting
   a) optionally an elution fraction 1 (EF1) in a side fraction container (SFC),
   b) an elution fraction 2 (EF2) in a product container, and
   c) optionally an elution fraction 3 (EF3) in a SFC,
wherein at least one of EF1 and EF3 is collected;
(4) loading on a chromatography matrix EF1 and/or EF3 and a second volume of the feed solution simultaneously or subsequently;
wherein steps (2) to (4) are repeated at least once, at least twice, at least 4 times, preferably at least 9 times, at least 14 times, more preferably at least 19 times, at least 24 times, most preferably at least 29 times; and
wherein the chromatography matrices of step (1) and (4) are the same or different chromatography matrices.

According to a preferred embodiment, the chromatography matrix of step 1 and 4 is the same first chromatography matrix; or the chromatography matrix of step 1 is a first chromatography matrix and the chromatography matrix of step (4) is a second chromatography matrix, and upon each repetition of steps (2) to (4), the chromatography matrices alternate between the second and the first chromatography matrix.

According to a further preferred embodiment, the volume of EF1 and/or EF3 collected in the SFC in step (3) is at least about 0.05, at least about 0.25, at least about 0.5, at least about 1, at least about 1.5, or at least about 2.0 volumes of the chromatography matrix.

According to a particular preferred embodiment, the concentration of an eluent comprised in the elution solution is increased over time during step (2) and wherein the eluent weakens the interaction between the product of interest and the chromatography matrix.

According to one embodiment, EF1 and/or EF3 comprise the product of interest and impurities, wherein compared to the solution loaded in step 1, the concentration of the product of interest is increased by a factor of at least about 2, preferably at least about 5, more preferably at least about 10. In this embodiment, the collection of EF1 is started at a first predetermined concentration X_{EF1-P} of the product of interest in the eluate and stopped at a predetermined concentration Y_{EF1-P} of the product of interest in the eluate and/or the collection of EF3 is started at a first predetermined concentration X_{EF3-P} of the product of interest in the eluate and stopped at a predetermined concentration Y_{EF3-P} of the product of interest in the eluate. In addition or alternatively, the collection of EF1 is started at a first predetermined concentration X_{EF1-I} of the impurities in the eluate and stopped at a predetermined concentration Y_{EF1-I} of the impurities in the eluate and/or the collection of EF3 is started at a first predetermined concentration X_{EF3-I} of the impurities in the eluate and stopped at a predetermined concentration Y_{EF3-I} of the impurities in the eluate. In addition to this embodiment or as an alternative to this embodiment of the present invention, EF2 comprises substantially pure product of interest, and the collection of EF2 is started at a predetermined concentration X_{EF2-P} of product of interest in the eluate and stopped at a predetermined concentration Y_{EF2-P} of product of interest in the eluate; and/or the collection of EF2 is started at a predetermined concentration X_{EF2-I} of impurities in the eluate and stopped at a predetermined concentration Y_{EF2-I} of impurities in the eluate.

According to a preferred embodiment, step (3) further comprises the step of diluting EF1 and/or EF3. The dilution preferably occurs in the SFC. Also preferably, EF1 and/or EF3 are diluted with the one or more of the feed, a chromatography buffer, and water.

According to one embodiment, the second volume of step (4) is the same as the first volume of step (1). According to an alternative embodiment, the second volume of step (4) is smaller than the first volume of step (1).

According to a particularly preferred embodiment, EF1 and/or EF3 collected from the first chromatography matrix are collected in a first SFC, and EF1 and/or EF3 collected from the second chromatography matrix are collected in a second SFC.

According to yet another embodiment, loading is stopped in steps (1) and (4) before any product of interest is eluted from the chromatography matrix with the flow-through.

According to one embodiment, steps (1) and (4) comprise binding the product of interest to the chromatography matrix.

According to one preferred embodiment, the chromatography matrices of step (1) and step (4) are of the same type.

According to another embodiment, the chromatography modus used in step (1) and step (4) is selected from the group consisting of a reversed-phase chromatography, a hydrophobic interaction chromatography, an affinity chromatography, an ion exchange chromatography, a cation exchange chromatography, an anion exchange chromatography, a mixed-mode chromatography, a chiral chromatography, a hydrophilic interaction liquid chromatography, a size exclusion chromatography and a dielectric chromatography.

According to a preferred embodiment, the chromatography modus of step (1) and step (4) is reversed-phase chromatography.

According to a preferred embodiment, the eluent comprised in the elution solution is a polar eluent. Preferably, the polar eluent is selected from the group consisting of acetonitrile, benzyl alcohol, methanol, acetic acid, ethylene glycol, tetrahydrofuran, ethanol, 1-propanol and 2-propanol.

According to a preferred embodiment, the chromatography matrices of step (1) and step (4) are chromatography columns.

According to a particular preferred embodiment, the product of interest is a polypeptide or protein.

According to one embodiment of the present invention, a chromatography apparatus comprises: one or more chromatography matrices with a first and a second end; a feed container; one or more a side fraction containers (SFC); conduit means connecting the second end of the one or more chromatography matrices with the one or more side fraction containers; conduit means connecting the one or more side fraction containers with the first end of the one or more chromatography matrices; conduit means connecting the feed container with the one or more side fraction containers; conduit means connecting the feed container with the first end of the one or more chromatography matrices. The one or more side fraction containers in this embodiment have a volume of about 0.05 to about 8 volumes of the chromatography matrices.

Further aspects and embodiments are disclosed in the accompanying claims and the following detailed description of the invention.

### BRIEF DESCRIPTION OF THE DRAWINGS

FIG. 1: Chromatogram of proteins eluting from a chromatographic column of the state of the art. The target component (red) is closely eluting with impurities (light blue and green). Dark blue lines indicate the sum signal as seen by the detector.

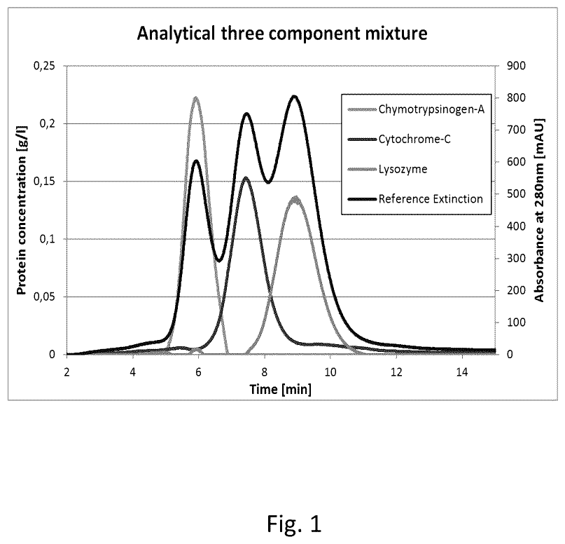

FIG. 2: Schematic presentation of a 6-column MCSGP operation described in Müller-Späth, 2014.

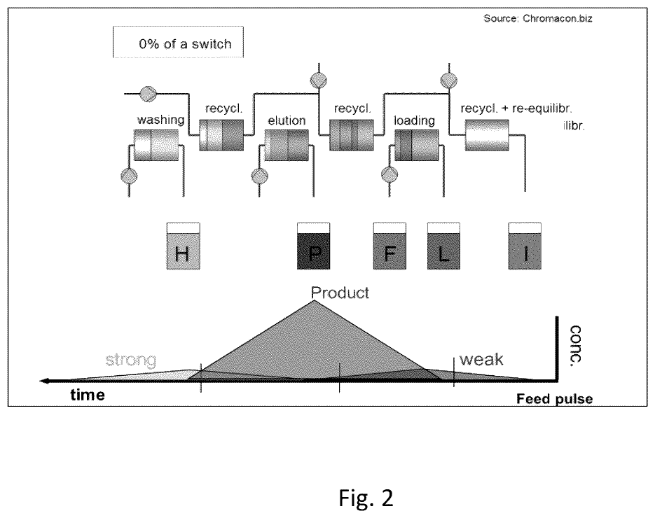

FIG. 3: Schematic presentation of the twin-column MCSGP steps throughout one cycle according to Krättli et al. (Journal of chromatography 2013, Vol. 1293, p. 51-59).

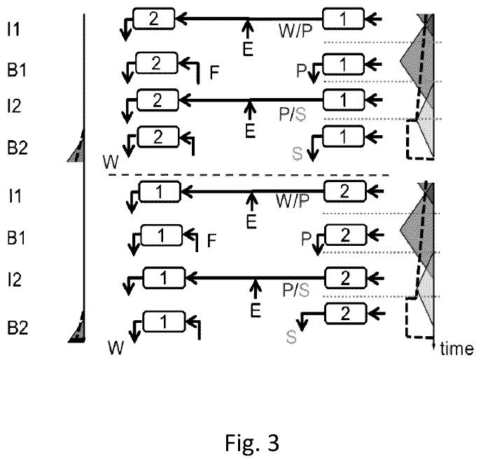

FIG. 4: Schedule of different process steps for conventional twin-column MCSGP. The scheme is divided at the y-axis value 0. The upper part shows feed loading (orange lines) and elution gradient (blue line) for column one. The lower part shows feed loading (light blue line) and elution gradient (yellow line) for the second column. The grey lines are the cut points for the overlap of product and impurities which are transferred from one column to the other as indicated by the red/grey arrows. Red arrows show product elution.

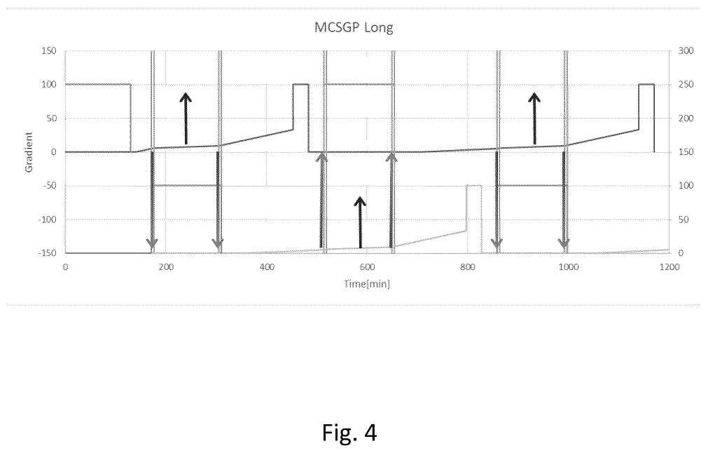

FIG. 5: Flow chart of the process of the present invention.

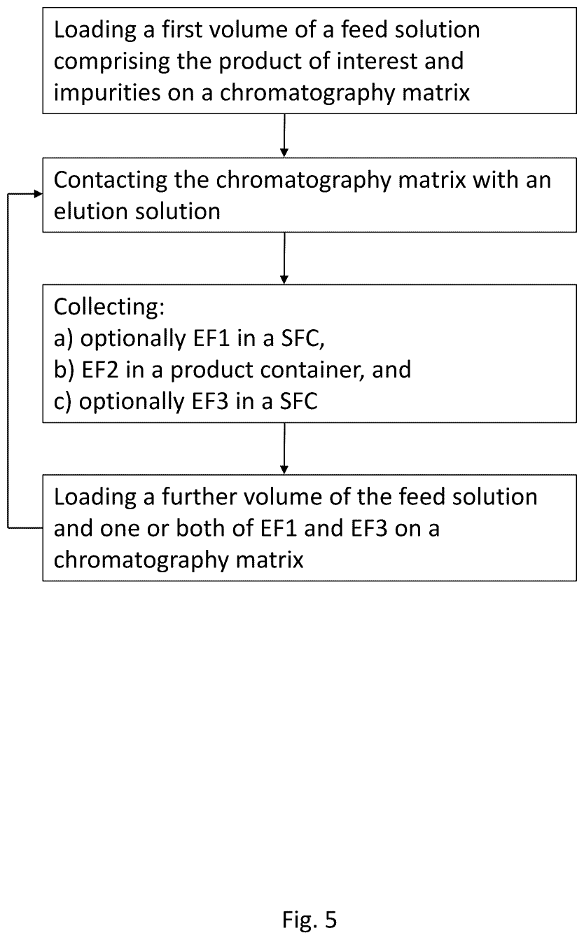

FIG. 6: Schematic representation of a preferred embodiment of the apparatus of the present invention with two matrices (columns 1 and 2) and two side fraction containers (P1, P2).

FIG. 7: Purity and yield for the method of the present invention over the number of cycles. Each cycle includes one repetition of steps (2) to (4), with the first round being denoted as X.1 and the repetition of steps (2) to (4) being denoted as X.2. The first cycle 1.1 included step (1), the following cycles 2 to 5 did not include step (1). Blue line indicates purity. Grey lines indicates yield of the cycle related to the amount of feed loaded. Yellow line indicates the overall yield. Orange line indicates yield in relation to the overall amount of proteins loaded (feed plus side fractions).

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Definitions

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, cell biology, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

As used herein, the term “and/or” means that it refers to either one or both/all of the options cited in the context of this term.

The terms “matrix” and “chromatography matrix” are used interchangeably herein and refer to the stationary phase in chromatography. The stationary phase can be present in form of a solid, a liquid, or a gel material, preferably in form of a resin or a combination of resins. The matrix can have the form of a column, a capillary tube, a plate, or a sheet. Particularly preferred is a chromatography matrix in form of a column. Preferred examples of a chromatography modus or method to be used in the context of the present invention include but are not limited to reversed-phase chromatography, hydrophobic interaction chromatography, affinity chromatography, ion exchange chromatography, cation exchange chromatography, anion exchange chromatography, mixed-mode chromatography, chiral chromatography, hydrophilic interaction liquid chromatography, size exclusion chromatography and dielectric chromatography. It is within the skilled person's competence to select a respective solid phase for the chromatography modus to be applied.

The terms “protein” and “polypeptide” are used interchangeably herein and refer to any peptide-bond-linked chain of amino acids, regardless of length or post-translational modification. Proteins usable in the present invention (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g. one or more of enhanced stability, increased biological half-life, or increased water solubility. Chemical modifications applicable to the variants usable in the present invention include without limitation: PEGylation, glycosylation of non-glycosylated parent polypeptides, covalent coupling to therapeutic small molecules, like glucagon-like peptide 1 agonists, including exenatide, albiglutide, taspoglutide, DPP4 inhibitors, incretin and liraglutide, or the modification of the glycosylation pattern present in the parent polypeptide. Such chemical modifications may occur co- or post-translational.

The term “amino acid” encompasses naturally occurring amino acids as well as amino acid derivatives. A hydrophobic non-aromatic amino acid in the context of the present invention, is preferably any amino acid which has a Kyte-Doolittle hydropathy index of higher than 0.5, more preferably of higher than 1.0, even more preferably of higher than 1.5 and is not aromatic. Preferably, a hydrophobic non-aromatic amino acid in the context of the present invention, is selected from the group consisting of the amino acids alanine (Kyte Doolittle hydropathy index 1.8), methionine (Kyte Doolittle hydropathy index 1.9), isoleucine (Kyte Doolittle hydropathy index 4.5), leucine Kyte Doolittle hydropathy index 3.8), and valine (Kyte Doolittle hydropathy index 4.2), or derivatives thereof having a Kyte Doolittle hydropathy index as defined above.

These descriptions and definitions are valid for the whole application unless it is otherwise stated.

### EMBODIMENTS

In the following, the elements of the present invention will be described. These elements are listed with specific embodiments, however, it should be understood that they may be combined in any manner and in any number to create additional embodiments. Specifically, embodiments described for the method of the present invention likewise apply to the apparatus of the present invention since the latter is designed for performing the method. The same applies for embodiments of the apparatus which may also be used in combination with the method of the present invention. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The present invention provides an improved chromatography method and an apparatus for performing the method. In the method of the present invention, for reducing product loss, the overlap of product and side component is captured and reloaded to increase yield.

According to a first aspect, the present invention provides a method of separating a product of interest from impurities comprising the following steps in the indicated order: (1) loading on a chromatography matrix a first volume of a feed solution comprising the product of interest and impurities; (2) contacting the chromatography matrix with an elution solution; (3) collecting a) optionally an elution fraction 1 (EF1) in a side fraction container (SFC), b) an elution fraction 2 (EF2) in a product container, and c) optionally an elution fraction 3 (EF3) in a SFC, wherein at least one of EF1 and EF3 is collected; and (4) loading a second or further volume of the feed solution simultaneously to or subsequently with EF1 and/or EF3 on a chromatography matrix. According to the method of the present invention, steps (2) to (4) are repeated at least once. According to a preferred embodiment of the present invention, steps (2) to (4) are repeated at least twice, at least 3 times, at least 4 times, or at least 5 times. Accordingly, steps 2 to 4 are repeated between 2 to 50 or more times, such as 3, 4, 5, 6, 7, 8, 9, 10, 11, 12, 13, 14, 15, 16, 17, 18, 19, 20, 21, 22, 23, 24, 25, 26, 27, 28, 29, 30 or more times, preferably at least 9 times, more preferably at least 14 times, even more preferably at least 19 times, yet even more preferably at least 24 times, and most preferably at least 29 times.

The present invention thus basically provides two setups for the method and the apparatus: the first setup comprises one chromatography matrix, and EF1 and/or EF3 are collected in one SFC before it is/they are fed to the matrix again. The second setup comprises two chromatography matrices, and EF1 and/or EF3 from the first matrix is/are collected in a first SFC before it is/they are fed to the second matrix, from which EF1 and/or EF3 is/are collected in a second SFC before it is/they are fed to the first matrix. Thus, according to one embodiment, a single chromatography matrix and a single side fraction container are used. According to an alternative embodiment, two chromatography matrices and two side fraction containers are used. It is also possible to use two chromatography matrices and a single side fraction container. Thus, according to one embodiment, the present invention provides a method and an apparatus, in which two chromatography matrices are used and EF1 and/or EF3 are collected in a single side fraction container.

According to a particularly preferred embodiment, EF1 (overlap of product and impurities before the main product elutes in EF2) is collected in the side fraction container (SFC), and EF3 (overlap of product and impurities after the main product elutes in EF2) is discarded. This discarding of EF3 can be performed in each repeating cycle of the method of the present invention, or alternatively in every other repeating cycle, in every third, in every fourth or in every fifth repeating cycle. According to a further embodiment of the present invention, EF1 is discarded and EF3 is collected in the side fraction container. This discarding of EF1 can be performed in each repeating cycle of the method of the present invention, or alternatively in every other repeating cycle, in every third, in every fourth or in every fifth repeating cycle. Therefore, the method of the present invention may additionally comprise a step of discarding EF1 and/or EF3. This discarding can be repeated according to the respective needs, as long as EF1 and/or EF3 are collected at least twice, at least three times, at least four times, at least five times or more when performing the method of the present invention. Discarding in the context of the present invention means not collecting the fraction in a side fraction container according to the present invention and not feeding the discarded fraction to any further chromatography matrix of the method or the apparatus of the present invention. In one particularly preferred embodiment, in the method of the present invention mainly EF1 is collected in a side fraction container and most of EF3 is discarded. More preferably, only EF1 is collected in a side fraction container. This preferred embodiment is particularly useful in cases where proteins are the product of interest.

According to the present invention, the chromatography matrices of steps (1) and (4) can be the same or different chromatography matrices. In the process of the present invention, the product is preferably bound to the chromatography matrix in steps (1) and (4).

The feed solution comprising the product of interest and impurities is preferably provided from a storage container.

In the embodiments using two chromatography matrices, these are preferably changed or switched after the first cycle of steps (1) to (4) of the method of the present invention. According to a preferred embodiment, upon each repetition of steps (2) to (4), the chromatography matrices alternate between the second the first chromatography matrix. In other words, a first matrix is loaded in step (1) with a first volume of a feed solution comprising the product of interest and impurities, and a second matrix is loaded in step (4) with elution fraction (EF) 1 and/or EF3 and a second volume of the feed solution. Upon repeating the steps (2) to (4), the second chromatography matrix loaded with EF 1 and/or EF3 and a second volume of the feed solution is eluted and—as described in steps (2) and (3)—EF2 is collected in a product container while EF1 and/or EF3 are transferred to a side fraction container, from which again the first matrix is loaded with EF1 and/or EF3 together with a volume of the feed solution.

According to one embodiment of the present invention, EF1 and/or EF3 collected from the first chromatography matrix are collected in a first SFC (P1), and EF1 and/or EF3 collected from the second chromatography matrix are collected in a separate second SFC (P2).

According to one embodiment of the present invention, EF1 and/or EF3 can be diluted. This dilution preferably takes place in step (3) and thus before EF1 and/or EF3 are loaded onto the chromatography matrix. Thus, according to a preferred embodiment, EF1 and/or EF3 are diluted in the SFC. EF1 and/or EF3 can be diluted with any suitable substance or composition. Preferably, EF1 and/or EF3 are diluted with the feed, a chromatography buffer, water, or any combination thereof. If water is used for dilution, the water is preferably de-ionized. Diluting EF1 and/or EF3 may prevent precipitation of product or other substances in EF1 and/or EF3.

According to a preferred embodiment, the second or further volume of the feed solution that is loaded onto the chromatography matrix in step (4) has the same volume as the first volume in step (1). Alternatively, the second or further volume of the feed solution that is loaded onto the chromatography matrix in step (4) is smaller than the first volume in step (1).

The product container, the storage container and the side fraction container(s) (SFC) can be any suitable container made from any suitable material known in the art. It is well within the skilled person's competence to decide which type of container is particularly suited for which separation process, and a number of suitable storage containers are commercially available. Since the present invention is not limited to separating any specific product or compound from impurities, the product container and the side fraction container(s) are not further defined. However, it is to be understood that the containers as used in the present invention are to be distinguished from a simple conduit. Thus, the containers of the present invention allow collection of a volume over the its axial length that is greater than the volume in a conduit of the same axial length. Preferably, a container of the present invention has a volume that is at least twice the volume of a conduit of the same length as the container. More preferably, the container has a volume that is at least 3 times, at least 4 times, at least 5 times, at least 6 times, at least 7 times, at least 8 times, at least 9 times or at least 10 times the volume of a conduit having the same length. A container as used in the context of the present invention is preferably connected to a chromatography matrix by one or more conduits. The terms “container” and “tank” are used interchangeably herein.

The chromatography matrices to be used in the present invention can be of the same type or of a different type (if two matrices are used). A chromatography matrix is selected depending on the individual needs and the product to be purified. The matrix can have the form of a column, a capillary tube, a plate, or a sheet. Particularly preferred is a chromatography matrix in form of a column. The chromatography matrices are preferably selected such that the product to be purified can bind to the matrix in steps (2) and (4) of the method of the present invention. The chromatography modes are preferably selected from the group consisting of reversed-phase chromatography, hydrophobic interaction chromatography, affinity chromatography, ion exchange chromatography, cation exchange chromatography, anion exchange chromatography, mixed-mode chromatography, chiral chromatography, hydrophilic interaction liquid chromatography, size exclusion chromatography, and dielectric chromatography. According to a preferred embodiment, the chromatography modus is reversed-phase chromatography. The chromatography matrices are preferably in the form of chromatography columns.

The present invention does not require any particular product to be separated and essentially any product can be separated and/or purified from impurities using the method and the apparatus of the present invention. A preferred product to be separated with the present method and apparatus, however, is a protein or polypeptide, preferably a recombinant protein or polypeptide expressed in a cell expression system. A further preferred product to be separated is a nucleic acid molecule, preferably mRNA.

According to one embodiment of the present invention, the volume of EF1 and/or EF3 collected in the SFC in step (3) is at least 0.05, at least 0.25, at least 0.5, at least 1, at least 1.5, or at least 2.0 the volume of the chromatography matrix.

According to a further preferred embodiment of the present invention, the concentration of an eluent comprised in the elution solution is increased over time during step (2). This allows a step-wise weakening of the interaction between the product of interest and the impurities with the chromatography matrix. Starting with a lower concentration of the eluent will at first release the weak binding impurities form the chromatography matrix. With increasing concentration of the eluate, the product of interest is preferably released from the matrix before also the strong binding impurities are eluated. Whether or not and when to increase the concentration of the eluent comprised in the elution solution will be determined by the skilled person on a case to case basis and will depend inter alia on the type of product to be separated/purified and the type of impurities. Thus, in some embodiments, the concentration of the eluent comprised in the elution solution is increased in one, two, three or more of the elution steps (2), while in other embodiments, the concentration of the eluent comprised in the elution solution is increased in essentially all elution steps (2) or not increased at all. It is within the competence of the skilled person to decide when the concentration of the eluent comprised in the elution solution is to be increased and to what degree.

When being eluted from the chromatography matrix, EF1 and/or EF3 comprise the product of interest and impurities. According to one embodiment, the absorption and/or concentration of the product of interest collected in the eluate is increased by a factor of at least 2 when compared to the feed solution loaded onto the chromatography matrix in step (1). According to a preferred embodiment, said concentration of the product of interest collected in the eluate is increased by a factor of at least 5, and more preferably of at least 10 when compared to the feed solution loaded onto the chromatography matrix in step (1). For starting the collection of EF1, EF2 and/or EF3, the absorption and/or concentration of the product or interest and/or the impurities at different stages of the method can be determined. For example, the collection of EF1 is preferably started at a first predetermined concentration/absorption X_{EF1-P} of the product of interest in the eluate, and stopped at a predetermined concentration/absorption Y_{EF1-P} of the product of interest in the eluate. In addition or alternatively, the collection of EF3 is started at a first predetermined concentration/absorption X_{EF3-P} of the product of interest in the eluate and stopped at a predetermined concentration/absorption Y_{EF3-P} of the product of interest in the eluate. In addition to determining the concentration/absorption of the product of interest, the concentration/absorption of the impurities can also be determined. Thus, the collection of EF1 is preferably started at a first predetermined concentration/absorption X_{EF1-I} of the impurities in the eluate, and stopped at a predetermined concentration/absorption Y_{EF1-I} of the impurities in the eluate. In addition or alternatively, the collection of EF3 is started at a first predetermined concentration/absorption X_{EF3-I} of the impurities in the eluate, and stopped at a predetermined concentration/absorption Y_{EF3-I} of the impurities in the eluate. In accordance with the present invention, EF2 preferably comprises product of interest. Thus, the collection of EF2 can be started a at predetermined concentration/absorption X_{EF2-P} of the product of interest in the eluate, and stopped at a predetermined concentration/absorption Y_{EF2-P} of product of interest in the eluate. As described above, for starting the collection of the individual fractions including EF2, the absorption and/or concentration of the product or interest and/or the impurities at different stages of the method can be determined. Thus, according to a further embodiment of the present invention, the collection of EF2 is started at a predetermined concentration/absorption X_{EF2-I} of impurities in the eluate, and stopped at a predetermined concentration/absorption Y_{EF2-I} of impurities in the eluate.

According to a preferred embodiment of the present invention, the loading in steps (1) and/or (4) is stopped before any product of interest is eluted from the chromatography matrix with the flow-through. This enables obtaining higher concentrations of the product of interest in higher purity and prevents loss of product in the overall process, contributing to an overall increase in efficiency.

The eluent comprised in the elution solution is preferably a polar eluent. According to a particularly preferred embodiment of the present invention, the eluent comprised in the elution solution is selected from the group consisting of acetonitrile, benzyl alcohol, methanol, acetic acid, ethylene glycol, tetrahydrofuran, ethanol, 1-propanol and 2-propanol.

According to an embodiment, the method comprises a step preceding step (1) of preparing the feed solution. Preferably, this preparation includes an initial separation or rough purification of the product of interest and may increase the concentration of the product of interest in the feed solution. The feed solution is thus preferably preferred by performing integrated counter current chromatography with a solution containing the product of interest. Integrated counter current chromatography is known to the skilled person as a combination of ion exchange and hydrophobic interaction chromatography. This step increases the concentration of the product in the feed solution.

According to a further embodiment of the present invention, concentrations of the product of interest and/or the impurities in the product container and the side fraction container(s), respectively, or at other points during the process or in the apparatus of the present invention are determined, for example by inline process analytical technologies. Determining the concentration of substances such as the product of interest and impurities is well within the competence of the skilled person and can be done, for example, by determining the absorption at a specific wavelength. Based on the absorption at a specific wavelength, the concentration of the respective substance can be calculated. Thus, according to one embodiment, the present invention comprises determining the absorption of the product of interest and/or of the impurities in the product container and the side fraction container(s), respectively, or at other points during the process or in the apparatus of the present invention.

The parameters for running the chromatography matrices depend on the type of product to be separated (product of interest) and may be set by the skilled person according to his/her experience and/or according to instructions of the manufacturers of e.g. the chromatography matrices and/or the solid phases used.

According to a particularly preferred embodiment, the present invention provides a chromatography apparatus comprising one or more chromatography matrices with a first and a second end, a feed container, one or more a side fraction containers (SFC), wherein the second end of the one or more chromatography matrices is in fluid connection with the one or more side fraction containers, wherein the one or more side fraction containers are in fluid connection with the first end of the one or more chromatography matrices, wherein the feed container is in fluid connection with the one or more side fraction containers, and wherein the feed container is in fluid connection with the first end of the one or more chromatography matrices. The fluid connection is preferably via conduit means. More preferably, the fluid connection can be regulated via one or more valve means. By using valves, the fluid flow can be directed between the individual components of the apparatus via the conduit means. According to a preferred embodiment, the one or more side fraction containers have a volume of about 0.05 to about 8 volumes of the chromatography matrices, such as about 0.1 to about 6, about 1 to about 4, about 2 to about 6, about 2 to about 4, and most preferably of about 2 to about 3 volumes of the chromatography matrices.

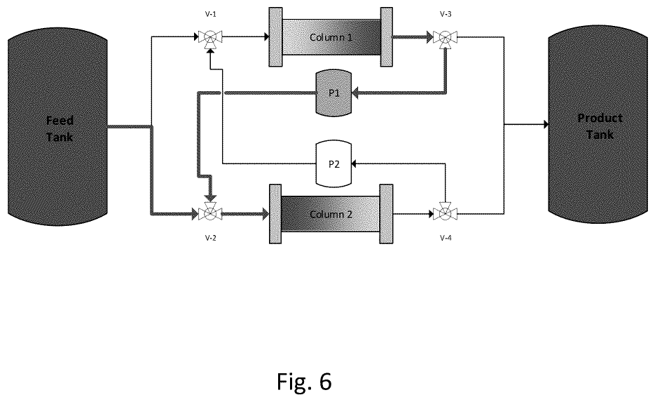

One preferred method and at the same time preferred apparatus of the present invention is schematically depicted in FIG. 6. In step (1), the feed solution comprising the product of interest and impurities is loaded from a feed tank to a first chromatography matrix (column 1). The loaded first chromatography matrix is then contacted in step (2) with an elution solution for obtaining in step (3) elution fractions 1 to 3. EF2 containing the product is led to a product container (product tank), and EF1 and/or EF3 are led to a first side fraction container (P1). In step (4), EF1 and/or EF3 from the first side fraction container (P1) are loaded either simultaneously with or subsequently to a further volume of the feed solution comprising the product of interest and impurities from the feed tank to the second chromatography matrix (column 2). This is the situation depicted in FIG. 6 with conduits under load being illustrated in a thicker way than conduits without any load. The process then continuous by repeating steps (2) to (4), however with switched roles of the first and the second chromatography matrices in that the loaded second chromatography matrix is contacted with an elution solution in step (2) and the elution fractions in step (3) are collected from said second chromatography matrix. Likewise, EF1 and/or EF3 are led to a second side fraction container (P2) from which they are led again to the first chromatography matrix on which they are loaded either simultaneously with or subsequently to a further volume of the feed solution comprising the product of interest and impurities from the feed tank to the first chromatography matrix, which starts another cycle of steps (2) to (4).

The present invention is characterized by using containers as time-buffers for the elution fractions in the method and apparatus, which allows the chromatography matrices to proceed with their steps and tasks individually. In this way, the overlap of product and impurity are not loaded directly onto the matrix but are stored in respective containers until the matrix can be loaded. The former synchronized steps can be performed separately, although the whole process stays synchronized in general throughout each cycle. The present invention thus preferably desynchronizes the individual steps by using side fraction containers, a storage container for the feed solution and a product container for the purified/separated product. The process provides an excellent performance in terms of purity and yield and at the same time gains further flexibility. Since the columns no longer need to wait for each other during the process steps, the process is faster and more productive. In addition, the desynchronization allows especially for continuous feed loading, which is a major benefit in chromatographic purification.

### EXAMPLES

The Examples are designed to further illustrate the present invention and serve a better understanding. They are not to be construed as limiting the scope of the invention in any way.

### Example 1

Chromatography was performed with a reversed phase resin (RP-resin) in self-packed Superformance® 600-16 columns (Götec-Labortechnik GmbH, Bickenbach, Germany). Bed height was set to 30 to 40 cm. An asymmetry of AS=1.44 was achieved. For hydrodynamic experiments, a Superformance® 150-10 column was packed to 10 cm bed height.

Feed was prepared out of frozen pool of recombinant protein which was mixed with deionized water in volumetric ratio 1:2 after defrosting. The deionized water was obtained from Arium® Pro (Sartorius Lab Instruments Gmbh & Co. KG, Göttingen, Germany).

Preparative runs were done with a LaPrep® system (VWR International, Radnor, PA, USA) consisting of two P110 pumps, one P314 UV detector and a Smartline 3900 auto sampler from Knauer (Knauer Wissenschaftliche Geräthe GmbH, Berlin, Germany). Peak fractionation was done with a Foxy Jr. sample collector (Teledyne Isco, Lincoln, NE, USA).

Analytical chromatographic separations were carried out with a VWR-Hitachi LaChrom Elite® system (VWR International, Radnor, PA, USA) with two high-pressure gradient pumps L-2130, L-2200 auto sampler, L-2350 column oven and L-2450 diode array detector (DAD).

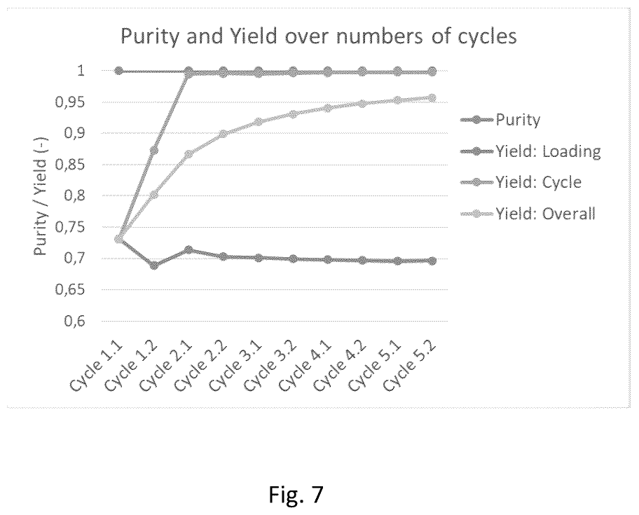

A two matrix column setup as schematically depicted in FIG. 6 has been used for purifying the recombinant protein. Purity and yield for the method of the present invention over the number of cycles (first cycle includes steps (1) to (4)=cycle 1.1, as well as repetition of steps (2) to (4)=cycle 1.2; the following cycles do not include step (1)) are shown in FIG. 7. Purity is maintained at high level while the overall yield increases with each cycle. Results for the setup of the present invention (“Continuous”) compared to results for the conventional method without feeding the side fractions to a chromatography matrix (“Batch”) are shown in table 1 below.

TABLE 1
Process parameters and results for the continuous gradient
elution chromatographic fractionation method (“Continuous”) compared
to a conventional method (“Batch”)

| | | Batch | Continuous | Deviation |
|---|---|---|---|---|
| Flow (feed) | l/min | 0.0095 | 0.0083 | −12.5% |
| Loading (per column) | l | 1.4 | 1.4 | 0% |
| Feed per day (max) | l | 6.27 | 11.97 | +91% |
| Feed per hour | l | 0.26 | 0.5 | +91% |
| (average) | | | | |
| Purity | [%] | 99.5 | 99.9 | |
| Yield | [%] | 73.1 | 95.8 | +31% |
| Production per day | g | 2.3 | 5.7 | +150.2% |
| Productivity | g/l/d | 33.51 | 41.9 | +25.1% |
| Eluent consumption | l/g | 1.78 | 1.36 | −23.6% |

As can be seen from table 1, the method and apparatus of the present invention lead to a significantly increased yield and thus productivity, while at the same time reducing eluent consumption compared to a conventional method.

1. A method of separating a product of interest from impurities comprising the following steps in the indicated order:
(1) loading on a chromatography matrix a first volume of a feed solution comprising the product of interest and impurities;
(2) contacting the chromatography matrix with an elution solution;
(3) collecting
a) optionally an elution fraction 1 (EF1) in a side fraction container (SFC),
b) an elution fraction 2 (EF2) in a product container, and
c) optionally an elution fraction 3 (EF3) in a SFC,
wherein at least one of EF1 and EF3 is collected;
(4) loading on a chromatography matrix EF1 and/or EF3 and a second volume of the feed solution simultaneously or subsequently;
wherein steps 2 to 4 are repeated at least once, at least twice, at least 4 times, preferably at least 9 times, at least 14 times, more preferably at least 19 times, at least 24 times, most preferably at least 29 times; and
wherein the chromatography matrices of step 1 and 4 are the same or different chromatography matrices.

2. The method of claim 1, wherein
(i) the chromatography matrix of step 1 and 4 is the same first chromatography matrix; or
(ii) the chromatography matrix of step 1 is a first chromatography matrix and the chromatography matrix of step 4 is a second chromatography matrix, and upon each repetition of steps 2 to 4, the chromatography matrices alternate between the second and the first chromatography matrix.

3. The method of claim 1 or 2, wherein the volume of EF1 and/or EF3 collected in the SFC in step 3 is at least 0.25, at least 0.5, at least 1, at least 1.5, or at least 2.0 volumes of the chromatography matrix.

4. The method of any of the preceding claims, wherein the concentration of an eluent comprised in the elution solution is increased over time during step 2 and wherein the eluent weakens the interaction between the product of interest and the chromatography matrix.

5. The method of any of the preceding claims, wherein
(i) EF1 and/or EF3 comprise the product of interest and impurities, wherein compared to the solution loaded in step 1, the concentration of the product of interest is increased by a factor of at least 2, preferably at least 5, more preferably at least 10, wherein
a) the collection of EF1 is started at a first predetermined concentration X_{EF1-P} of the product of interest in the eluate and stopped at a predetermined concentration Y_{EF1-P} of the product of interest in the eluate and/or the collection of EF3 is started at a first predetermined concentration X_{EF3-P} of the product of interest in the eluate and stopped at a predetermined concentration Y_{EF3-P} of the product of interest in the eluate; and/or
b) the collection of EF1 is started at a first predetermined concentration X_{EF1-I} of the impurities in the eluate and stopped at a predetermined concentration Y_{EF1-I} of the impurities in the eluate and/or the collection of EF3 is started at a first predetermined concentration X_{EF3-I} of the impurities in the eluate and stopped at a predetermined concentration Y_{EF3-I} of the impurities in the eluate; and/or
(ii) EF2 comprises substantially pure product of interest, wherein
a) the collection of EF2 is started at a predetermined concentration X_{EF2-P} of product of interest in the eluate and stopped at a predetermined concentration Y_{EF2-P} of product of interest in the eluate; and/or
b) the collection of EF2 is started at a predetermined concentration X_{EF2-I} of impurities in the eluate and stopped at a predetermined concentration Y_{EF2-I} of impurities in the eluate.

6. The method of any of the preceding claims, wherein step 3 further comprises the step of diluting EF1 and/or EF3, preferably wherein the dilution occurs in the SFC, and/or
wherein EF1 and/or EF3 are diluted with the one or more of the feed, a chromatography buffer, and water.

7. The method of any of the preceding claims, wherein
(i) the second volume of step 4 is the same as the first volume of step 1, or
(ii) the second volume of step 4 is smaller than the first volume of step 1.

8. The method of any of claims 2 to 7, wherein EF1 and/or EF3 collected from the first chromatography matrix are collected in a first SFC, and EF1 and/or EF3 collected from the second chromatography matrix are collected in a second SFC.

9. The method of any of the preceding claims,
wherein in steps 1 and 4 loading is stopped before any product of interest is eluted from the chromatography matrix with the flow-through;
wherein steps 1 and 4 comprise binding the product of interest to the chromatography matrix; and/or
wherein the chromatography matrices of step 1 and step 4 are of the same type.

10. The method of any of the preceding claims, wherein the chromatography modus of step 1 and step 4 is selected from the group consisting of a reversed-phase chromatography, a hydrophobic interaction chromatography, an affinity chromatography, an ion exchange chromatography, a cation exchange chromatography, an exchange anion chromatography, a mixed-mode chromatography, a chiral chromatography, a hydrophilic interaction liquid chromatography, a size exclusion chromatography and a dielectric chromatography.

11. The method of any of the preceding claims, wherein the chromatography matrices of step 1 and step 4 are reversed-phase chromatography matrices.

12. The method of claim 11, wherein the eluent comprised in the elution solution is a polar eluent, in particular selected from the group consisting of acetonitrile, benzyl alcohol, methanol, acetic acid, ethylene glycol, tetrahydrofuran, ethanol, 1-propanol and 2-propanol.

13. The method of any of the preceding claims, wherein the chromatography matrices of step 1 and step 4 are chromatography columns.

14. The method of any of the preceding claims, wherein the product of interest is a polypeptide or protein.

15. A chromatography apparatus comprising
one or more chromatography matrices with a first and a second end;
a feed container;
one or more a side fraction containers (SFC);
conduit means connecting the second end of the one or more chromatography matrices with the one or more side fraction containers;
conduit means connecting the one or more side fraction containers with the first end of the one or more chromatography matrices;
conduit means connecting the feed container with the one or more side fraction containers;
conduit means connecting the feed container with the first end of the one or more chromatography matrices;
wherein the one or more side fraction containers have a volume of about 0.05 to about 8 volumes of the chromatography matrices.
